(19) United States
(12) Patent Application Publication
(10) Pub. No.: US 2023/0016885 A1
(43) Pub. Date: Jan. 19, 2023

(54) Broach Handle

(71) Applicant: Invictus Orthopaedics LLC, Teaneck, NJ (US)

(72) Inventors: Kenneth McCulloch, Manhasset, NY (US); Anthony D'Antuono, South Amboy, NJ (US); Troy Lane, Murrells Inlet, SC (US)

(73) Assignee: Invictus Orthopaedics LLC, Teaneck, NJ (US)

(21) Appl. No.: 17/697,765

(22) Filed: Mar. 17, 2022

Related U.S. Application Data
(63) Continuation-in-part of application No. 16/691,271, filed on Nov. 21, 2019.
Application No. 16/691,271 is a continuation of application No. 15/627,723, filed on Jun. 20, 2017, now abandoned.
(60) Provisional application No. 63/297,947, filed on Jan. 10, 2022.
(60) Provisional application No. 62/353,261, filed on Jun. 22, 2016.
(60) Provisional application No. 62/510,111, filed on May 23, 2017.

(51) Int. Cl. A61B 17/16; A61F 2/46

(52) U.S. Cl. CPC A61B 17/1668; A61B 17/164; A61F 2/4607; A61B 2017/0046

(57) ABSTRACT

A surgical instrument for releasably connecting to a surgical tool, having three main sections that are able to articulate 360 degrees in 60-degree increments. The articulation of the sections allows for the distal end of the surgical instrument to be spatially offset from the proximal end, yet maintain parallel longitudinal axes. Varying surgical tools can be connected to the surgical instrument via an adapter.

### CROSS REFERENCE TO RELATED APPLICATIONS

The present application claims the benefit of U.S. Provisional Application No. 63/297,947 filed on Jan. 10, 2022, and is a Continuation-in-Part of U.S. patent application Ser. No. 16/691,271, filed on Nov. 21, 2019, which is a Continuation of U.S. patent application Ser. No. 15/627,723, filed on Jun. 20, 2017 (now abandoned), which claims the benefit of U.S. Provisional Application No. 62/353,261, filed on Jun. 22, 2016, and U.S. Provisional Application No. 62/510,111, filed on May 23, 2017. Priority is hereby claimed to each of those former applications, and the entire contents of each of those applications are incorporated herein by reference in their entireties as if set forth fully herein.

### FIELD

Example aspects here generally relate to surgical instruments used in minimal incision surgery aiding in the installation of orthopedic prosthesis, and more particularly, to a surgical instrument having a releasable connection to another surgical tool for preparing a bone site prior to the implantation of a hip prosthesis' femoral component during hip replacement surgery.

### BACKGROUND

Joint implants, also referred to as, for example, joint prostheses or joint replacements, are long-term surgically implantable devices that are used to partially or totally replace diseased or damaged joints, such as a hip, a knee, a shoulder, an ankle, or an elbow, within the musculoskeletal system of a human or an animal. Artificial hip joints are generally ball and socket joints, designed to match as closely as possible the function of the natural joint. Generally, the artificial socket is implanted in one bone, and the artificial ball articulates in the socket. A stem structure attached to the ball is implanted in another of the patient's bones, securing the ball in position.

The ball and socket joint of the human hip unites the femur to the pelvis. The head of the femur or ball fits into the acetabulum of the pelvis, forming a joint which allows the leg to move forward, backward, and sideways in a wide range.

Various degenerative diseases and injuries may necessitate replacement of all or a portion of a hip using synthetic materials. Prosthetic components are generally made from metals, ceramics, or plastics, or combinations of them.

Total hip arthroplasty and hemi-arthroplasty are two well-known procedures for replacing all or part of a patient's hip. A total hip arthroplasty replaces both the femoral component and the acetabular surface of the joint. A hemi-arthroplasty may replace either the femoral component or the acetabular surface of the joint. The purpose of hip replacement surgery is to remove the damaged and wom parts of the hip and replace them with artificial parts, called prostheses, with the purpose of at least partially restoring the hip's function, including but not limited to, restoring the stability, strength, range of motion, and flexibility of the joint.

In total hip replacement surgery, a patient's natural hip is replaced by two main components: an acetabular cup component that replaces the acetabular socket, and the femoral component, or the stem-and-ball component that replaces the femoral head.

In order to install the acetabular cup, a surgeon prepares the bone by reaming the acetabular socket to create a surface for accepting a cup. The cup may be held in place by bone cement or an interference or press fit, or it may have a porous outer surface suitable for bony ingrowth. The new acetabular shell is implanted securely within the prepared hemispherical socket.

Next, the femur is prepared to receive the stem. The proximal end of the femur is at least partially resected to expose the central portion of the bone. In the central portion, a cavity is created that matches the shape of the implant stem. The top end of the femur is planed and smoothed so that the stem can be inserted flush with the bone surface.

It is highly desirable to adapt the surgical instruments used in preparation of the femoral bone during hip replacement to minimally invasive surgery, computer assisted surgery, or both. The instruments used in femoral preparation include, but are not limited to, osteotomes or chisels used for resecting at least a portion of the femoral head to expose the central portion of the femur, and broaches, reamers, and rasps, used to clean and enlarge the hollow center of the bone, creating a cavity that matches the shape of the femoral component's stem.

During hip replacement surgery, the surgeon opens a femoral intramedullary canal by removing a portion of the trochanteric fossa with an osteotome or a chisel, an instrument for surgical division or sectioning of bone. The surgeon then uses one or a series of increasing size cavity preparation devices, such as reamers or broaches, to prepare a cavity for installation of a femoral stem. By using a series of gradually increasing in size devices, the surgeon expands the intra-femoral cavity until the desired size and shape is created. Sometimes, the portion of the final broach inserted into the femoral cavity serves as a trial femoral stem.

It is generally desired to select and install the femoral stem of the largest size suitable for a particular patient. Electing the largest appropriate femoral stem helps to stabilize the femoral component in the femur, improves alignment, and reduces the potential of the femoral component's loosening and failure. There is a need for instruments and method for preparation of a femoral cavity that permit installation of an appropriately sized stem of the femoral component in order to improve alignment and stabilization of the femoral component in the patient with minimum interference the tissue of the patient.

In minimally invasive surgery, the need to insert and operate the femoral preparation instruments through smaller incisions may conflict with the proper instrument alignment needed to create the cavity of the largest possible size. For proper access and alignment, long incisions and other invasive procedures are often required. The single-incision lateral or posterior approach hip-arthroplasty procedure may simplify access to the femur. A direct anterior approach for minimally invasive total hip arthroplasty has become increasingly popular. Preparation of the femoral canal using this approach can be technically challenging. Instrumentation of the femur involves a posteromedial capsular release, extension and external rotation of the operative leg and elevation of the femur anteriorly. Curved offset femoral broaches have been specifically designed to safely prepare the femoral canal through this single incision. A broach handle with lateral and anterior offset for the direct anterior approach has been developed to reduce the need for leverage of the proximal femur for preparation of the cavity.

Because a surgeon may perform a left hip replacement surgery or a right hip replacement surgery, it is currently necessary that the surgeon have both left and right lateral offset broach handles. Similarly, depending on a patient's body type, i.e., large protruding mid-section, current offset broach handles may not have adequate offset to account for such patients. Thus, it is necessary to have a broach handle that is adaptable to different situations and conditions.

In summary, there is a current unrealized need for improved devices, systems and procedures adapted for use in minimally invasive surgery (MIS). There is a particular unrealized need for improved devices for preparation of a patient's femur for installing a femoral component of a hip prosthesis. Improved devices are desired that are adapted for introduction and operation through a smaller surgical incision than conventionally available devices. Also needed are improved devices, systems, and procedures that would minimize the damage to the flesh, muscle, and other soft tissues during insertion, operation, and withdrawal. At the same time, there is a need for improved devices, systems, and procedures that would improve sizing and aligning of the femoral components and reduce the risk of their loosening In general, devices and systems are needed that are easy and reliable to use and manufacture, minimizing tissue damage, simplifying surgical procedures, and are versatile, allowing for faster healing with fewer complications, requiring less post-surgical immobilization, and are less costly to produce and operate, as our instrument replaces the need for 3 handles; providing left, right and straight approach.

### SUMMARY

The foregoing and other problems and deficiencies in known broach handles are solved and a technical advantage is achieved by an articulating broach handle.

An example embodiment herein is an articulating surgical tool handle, comprising a force disc, three main sections, two articulating means, and means for releasably attaching a tool. Wherein the articulating means allow each of the three sections to rotate 360 degrees with respect to the other sections.

In a further example embodiment herein the two articulating means allows articulation of the two or more sections in discrete increments.

In another example embodiment herein the three main sections comprise a Main Grip, a Center Body

Section, a Broach Connect, and the two articulating means allow for a double offset left, double offset right and straight configuration.

In still a further example embodiment herein, in the double offset configuration, the Main Grip is offset spatially from the Broach and maintain parallel axes and compound axial configuration of the longitudinal Main Grip and Broach Connect Body.

In another example embodiment herein, the spatial offset of the Main and Broach Connect Body is adjustable by two articulating means.

In yet another example embodiment herein the discrete increments are of the range of 60 degree incremental rotation respectively to each joint.

There is also the ability to provide a compound angle off-set, wherein you can exceed the parallel axial embodiments of main body and connecting body to acquire the desired positional attitude for non-invasive surgery.

According to another example embodiment herein, the articulating means comprises a Push Button, wherein the Push Button allows for the adjustment of the position of the two or more sections relative to each other, and the cam mechanism allows for the two or more sections to be in a fixed position relative to the other of the two or more sections.

According to an example embodiment herein, a method is provided for using an articulating surgical tool handle to prepare a patient's femur for installing a prosthetic stem component into the medullary canal of the femur. The method comprises the steps of providing an articulating surgical tool handle to attach a broach for installing a prosthetic stem component. A surgeon determines whether the left or right hip is to operated on. Depending on which side, the surgeon configures the articulating surgical tool for that particular side. The articulating surgical tool handle comprises a force disc, a main grip (Main longitudinal section and force disc can be one piece), a center body section, a broach connect body section, a first articulating means connecting the main grip section and the center body section, a second articulating means connecting the center body section and the broach connect body section, and a broach connected to the broach connect body section via an adapter. The method further includes the steps of attaching a broach to the broach connect body section via the adapter, ensuring the engraving indicators align with each other, adjusting the first and second articulating means to spatially offset the main grip section from the broach connect body section, inserting the articulating surgical tool handle and broach into the medullary canal through a surgical incision, positioning the articulating surgical tool handle and broach, preparing the medullary canal by striking the force disc to create a femoral canal, and removing the articulating surgical tool handle and broach from the medullary canal. The articulating surgical tool handle can be configured for either a left or right hip replacement surgery, and depending on the physical characteristics of a patient, the spatial offset between the main longitudinal section and the broach section can be adjusted using one or both of the articulating means, (including a straight approach).

Other devices, apparatuses, methods, features, and advantages herein will be, or will become, apparent to one with skill in the art upon examination of the following figures and detailed description. It is intended that all such additional systems, methods, features, and advantages be included within this description, be within the scope of the invention, and be protected by the following claims.

### BRIEF DESCRIPTION OF THE DRAWINGS

The foregoing summary, as well as the following detailed description of the invention will be better understood when read in conjunction with the appended drawings. It should be understood, however, that the invention can be embodied in different forms and thus should not be construed as being limited to the embodiments set forth herein.

FIG. 1 is an exploded, perspective view of an embodiment of an articulating broach handle;

FIG. 2 is a top view of the Main Grip section of the articulating broach handle of FIG. 1;

FIG. 3 is a left, side view of the Main Grip section of the articulating broach handle of FIG. 1;

FIG. 4 is an isometric view of the Main Grip section of the articulating broach handle of FIG. 1;

FIG. 5 is as top view of the Center Body section of the articulating broach handle of FIG. 1;

FIG. 6 is a left, side view of the Center Body section of the articulating broach handle of FIG. 1;

FIG. 7 is a perspective view of the Center Body section of the articulating broach handle of FIG. 1;

FIG. 8 is an isometric view of the Conical Compression Spring of the articulating broach handle of FIG. 1.

FIG. 9 is an isometric view of the Dowel of the articulating broach handle of FIG. 1;

FIG. 10 is a top view of the Dowel of FIG. 9;

FIG. 11 is a left, side view of the Dowel of FIG. 9;

FIG. 12 is a front view of the Dowel of FIG. 9;

FIG. 13 is an isometric view of the Dowel of FIG. 9;

FIG. 14 is aback view of the Broach Connect Body section of the articulating broach handle of FIG. 1;

FIG. 15 is atop view of the Broach Connect Body section of the articulating broach handle of FIG. 1;

FIG. 16 is a front view of the Broach Connect Body section of the articulating broach handle of FIG. 1;

FIG. 17 is an isometric view of the Broach Connect Body section of the articulating broach handle of FIG. 1;

FIG. 18 is an isometric view of the Pincher Dowel of the articulating broach handle of FIG. 1;

FIG. 19 is a top view of the Lock Pin of the articulating broach handle of FIG. 1;

FIG. 20 is a front, side view of the Lock Pin of the articulating broach handle of FIG. 1;

FIG. 21 is a left, side view of the Lock Pin of the articulating broach handle of FIG. 1;

FIG. 22 is an isometric view of the Lock Pin of the articulating broach handle of FIG. 1;

FIG. 23 is a right, side view of the Lock Lever of the articulating broach handle of FIG. 1;

FIG. 24 is a top view of the Lock Lever of the articulating broach handle of FIG. 1;

FIG. 25 is an isometric view of the Lock Lever of the articulating broach handle of FIG. 1;

FIG. 26 is a front view of the Adapter of the articulating broach handle of FIG. 1;

FIG. 27 is a top view of the Adapter of the articulating broach handle of FIG. 1;

FIG. 28 is an isometric view of the Adapter of the articulating broach handle of FIG. 1;

FIG. 29 is an isometric view of the Adapter of the articulating broach handle of FIG. 1

FIG. 30 is a top view of the Left Pincher of the articulating broach handle of FIG. 1;

FIG. 31 is a left, side view of the Left Pincher of the articulating broach handle of FIG. 1;

FIG. 32 is an isometric view of the Left Pincher of the articulating broach handle of FIG. 1;

FIG. 33 is a bottom view of the Right Pincher of the articulating broach handle of FIG. 1;

FIG. 34 is a side view of the Right Pincher of the articulating broach handle of FIG. 1;

FIG. 35 is an isometric view of the Right Pincher of the articulating broach handle of FIG. 1;

FIG. 36 is an isometric view of a Broach;

FIG. 37 is an isometric view of the Compression Spring of the articulating broach handle of FIG. 1;

FIG. 38 is an exposed view of the Push Button mechanism;

FIG. 39 is an isometric view of the Push Button mechanism in the disengaged position;

FIG. 40 is an isometric view of the Push Button mechanism in the engaged position;

FIG. 41 is the Broach Handle is the neutral Left alignment;

FIG. 42 is the Broach Handle is the neutral Right alignment;

FIG. 43 is a cutaway of the Broach to the Broach Handle.

### DETAILED DESCRIPTION

The present subject matter will now be described more fully hereinafter with reference to the accompanying figures, in which representative embodiments are shown. The present subject matter can, however, be embodied in different forms and should not be construed as limited to the embodiments set forth herein. Rather, these embodiments are provided to describe and enable one of skill in the art. Unless otherwise defined, all technical and scientific terms used herein have the same meaning as commonly understood by one of ordinary skill in the art to which the subject matter pertains. All publications, patent applications, patents, and other references mentioned herein are incorporated by reference in their entirety.

The attached Appendix 1 (33 sheets) and Appendix 2 (92 sheets) are considered parts of the present application and are therefore incorporated by reference herein in their entireties, as if set forth fully herein.

Example embodiments herein relate to an articulating broach handle with a releasable connection to a broach that has three sections that articulate 360 degrees with respect to each other. The specification describes a fifth prophetic embodiment. Embodiments 1 through 4 are described in Applicants' previous patent applications (USPTO Serial Nos. 16/691,271 and 15/627,723). Each embodiment is composed of hardened 420 stainless steel, hardened 440 steel and/or titanium. It is also contemplated that the inner components are constructed from either hardened 420 or hardened 440 stainless steel and the external components are constructed of titanium. However, other forms of surgical steel may be used; (304SS, 304L SS, 316SS, 420SS, 440SS, 430SS, 434SS). One of ordinary skill in the art will understand that the description of the invention is not limited to a broach handle.

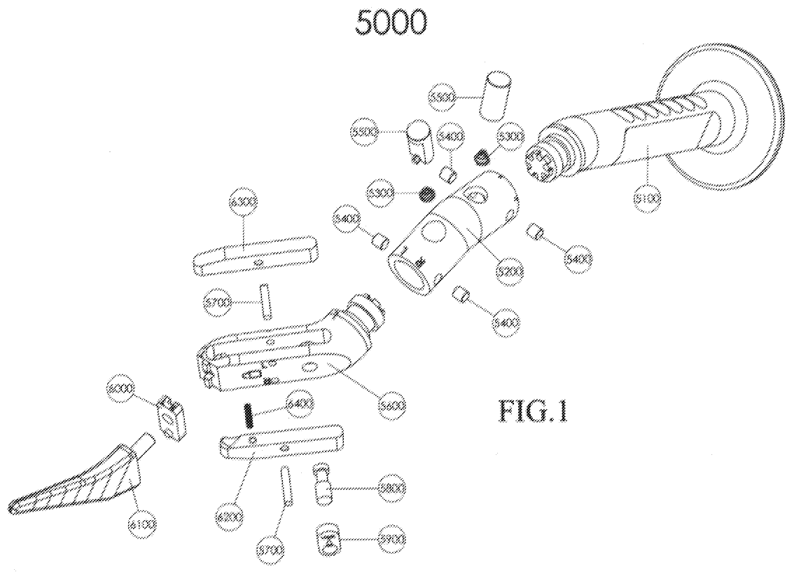

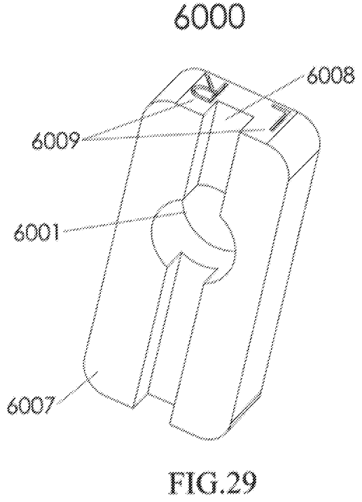

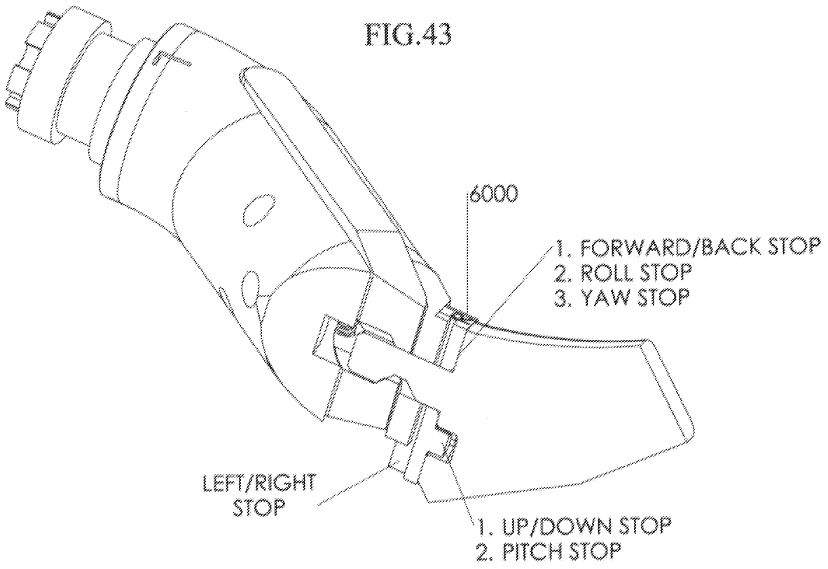

FIGS. 1 through 44 depict example embodiment(s) herein as described in the context of a Broach Handle 5000. The broach handle from the proximal end to the distal end consists of three main sections, a Main Grip 5100, a Center Body 5200, a Broach Connect Body 5600 together with an Adapter 6000. A Broach 6100, not part of the present invention, may be a commercially available broach from one of many manufacturers that connects to the Broach Connect Body 5600 via Adapter 6000.

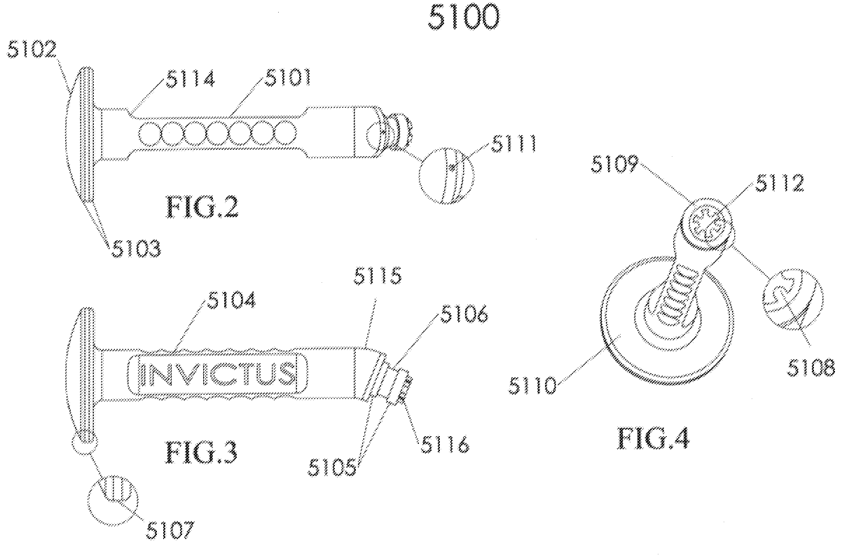

The Main Grip 5100, depicted in FIGS. 2 through 4 includes a circular shaped Force Disc 5110 and may have chamfered or radial fillet edges 5103. The diameter of the Force Disc is preferably 2.5 inches and a total thickness of 0.75 inches, comprising an arcing dome surface (convex surface) 5102 projecting approximately 0.3 inches from a uniform side 5110 having a thickness of approximately 0.4 inches. The arcing dome surface serves as an infinite vectoring force distribution for directional force, allowing for decisive directional device travel upon impact end-user impact. The proximal surface 5102 is convex with a radius of approximately 2.27 inches. The distal or impact side 5110 of the Force Disc provides a planer surface for Broach removal via a proximal impact. The Force Disc may be permanently or semi-permanently affixed to the shaft of the Main Grip.

The Main Grip 5100 has a longitudinal linear shaft 5114 with a Main Grip Bend 5115 near the distal end 5116. The linear shaft includes mirrored Gripping Notches 5101 within the outer diameter of the linear (Main Grip) shaft for added instrument grip and provides overall weight reduction of the instrument. The linear shaft also includes mirrored scallops or radial serrations 5104 providing further instrument gripping capability. Preferably there are 14 (7 mirrored scallops, 14 total) mirrored scallops or radial serrations. It is envisioned that the number of mirrored scallops or radial serrations may vary. At the distal end 5116 of the Main Grip are two Main Grip Mating Surfaces 5105 which provide concentric alignment with the Center Body Bore 5204 (discussed down below). In between the Main Grip Mating Surfaces is a Main Grip Connecting Groove 5106 which provides linear joint attachment with the Center Body 5200 via Dowel 5400, allowing for 360-degree rotation. Proximal the Main Grip Mating Surfaces is a Positional Identifier 5111, a circular bore recess, for 60-degree incremental rotation with the Positional Identifier 5201 of the Center Body 5200. The distal end 5116 includes a Main Grip Thrust Face 5109 which provides a coincident planer surface for mating with the Center Body Mating Thrust Face 5205. The distal end of the Main Grip further includes a Main Grip Outside Diameter 5112 provides clearance for the Button Second Planer Surface 5507 of the Button Engaging Member 5509 of the Push Button 5500 and has six positional slots 5108.

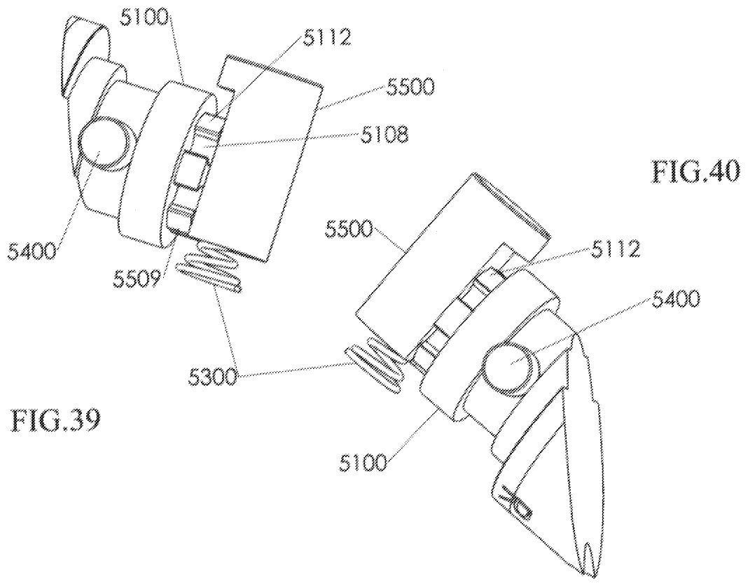

When the Button Push 5500 is pressed (see FIG. 39), Conical Spring 5300 is compressed, Button Engaging Member 5509 disengages from Positional slot 5108 allowing the Center Body to rotate in 60-degree increments for 360-degree rotation. When the Push Button 5500 is released or in the engaged position (FIG. 40), Conical Spring 5300 is no longer compressed and applying a force to allow a Positional Slot to engage with the Button Angled Locking Surfaces 5505 of the Button Engaging Member 5509 preventing rotation. This function is similar to the rotation of the Center Body 5200 with the Broach Connect Body 5600.

The bend 5115 is preferably between 50 and 80 degrees, and more preferably 67.5 degrees. The length of the Main Grip 5100 from the proximal end 5117 to the Bend 5115 is approximately 6.0 inches. From the Bend 5115 to the absolute distal end 5116 is approximately 1.0 inch.

The Main Grip distal end 5116 at the bend 5115 and beyond in the distal direction is cylindrical. The diameter of the Bend is 1.125″ and the absolute distal end has a diameter of 0.619″ inches. Also, a dual joining surface diameter of 0.796″ that slip fits within the adjoining connective bore of center body, proximally and distally. The above measurements and those following are exemplary figures. Other dimensions are contemplated.

The Center Body 5200 (FIGS. 5 through 7) of the Broach Handle 5000 interconnects the Main Grip 5100 and the Broach Connect Body 5600. The Center Body 5200 comprises a proximal end 5214 which is angled at 22.5 degrees from the distal end 5215. The Center Body provides 2 Rotational Joints 5218 and 5219 and 2 Push Buttons 5500. (FIG. 1)

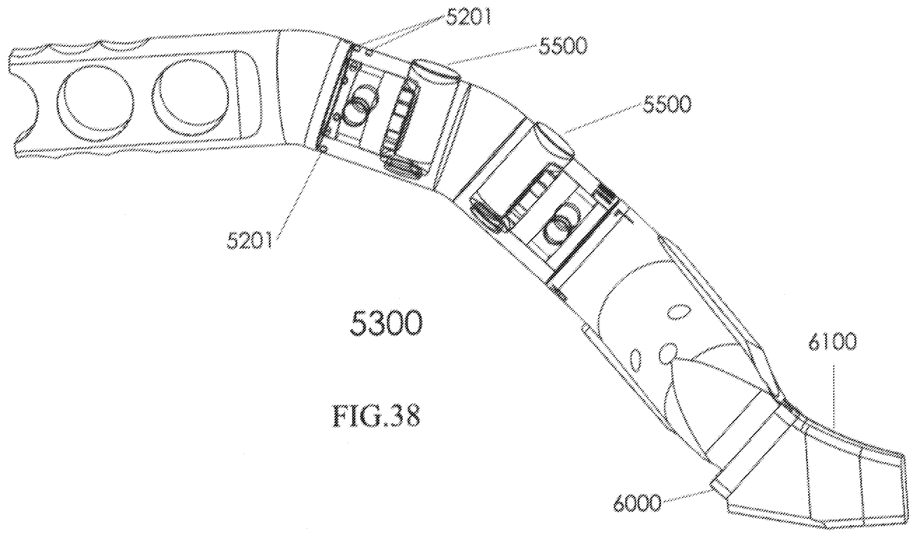

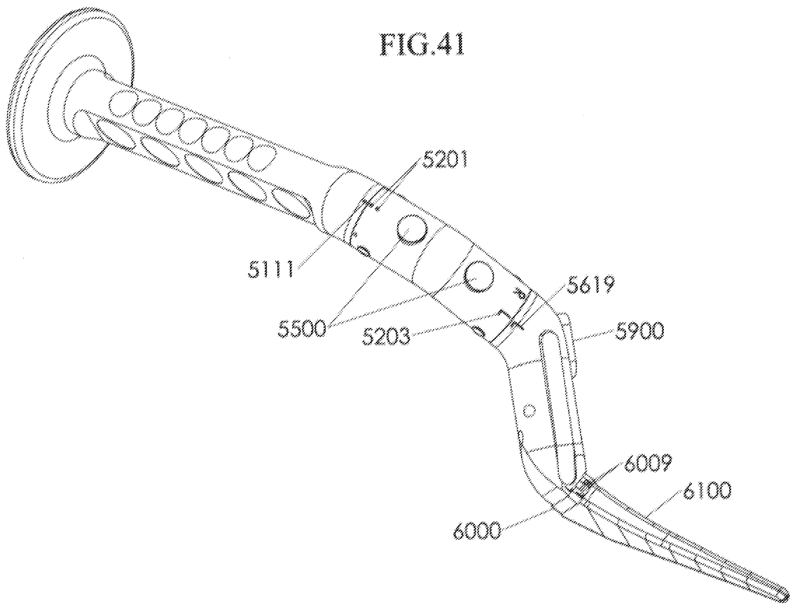

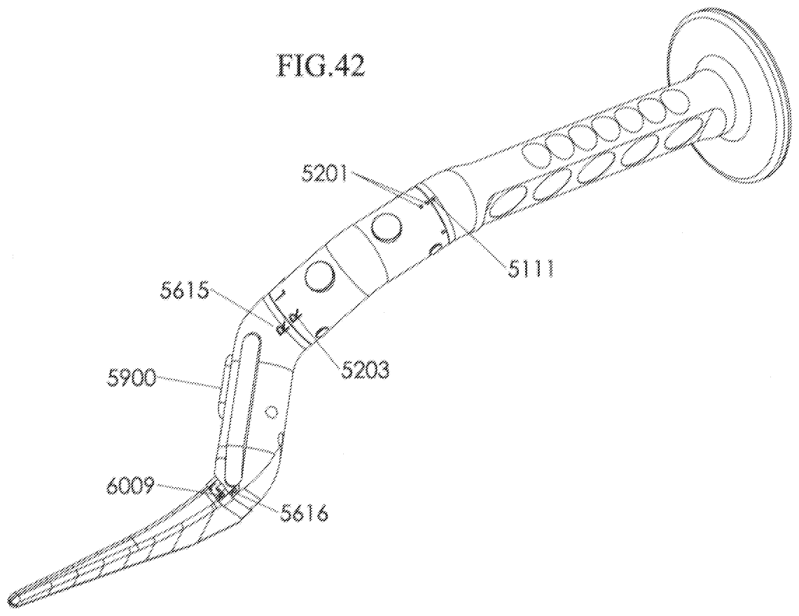

The proximal end 5214 includes six Positional Identifiers 5201. The Positional Identifiers are circular bored recesses for referencing 60-degree incremental positional rotations and align with the Positional Identifier 5111 on the Main Grip 5100 depending on the angle of rotation. At one position on the Center Body, there are two Positional Identifiers in line (see FIGS. 38, 41 and 42). When the Positional Identifier 5111 on the Main Grip aligns with the 2 Positional Identifiers on the Center Body (see FIGS. 41 and 42), the Broach Handle is in the neutral position (locked position for either offset, Left or Right at zero (0) degrees rotation). At the distal end 5215 are engravings 5203 (R and L) for detailed position of Left and Right positions.

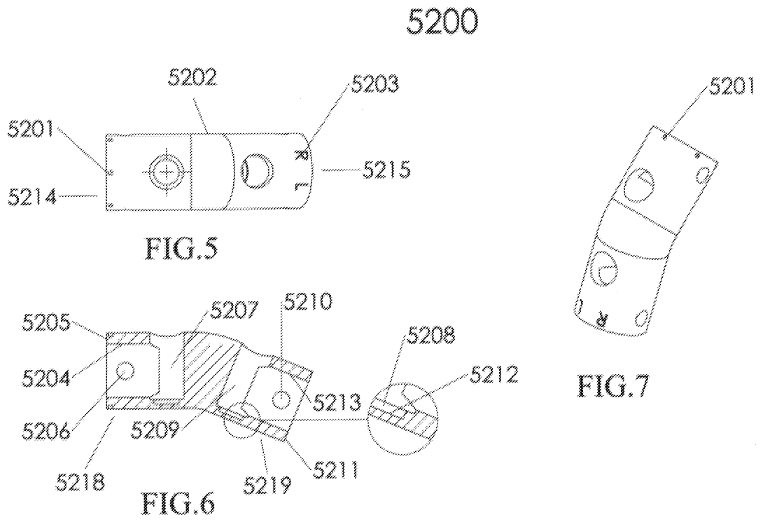

The Center Body Outer Diameter Geometry 5202 provides the outer boundary geometry for anatomical clearance. As noted previously, the geometry of the Center Body exhibits a symmetrical 22.5-degree angle (FIG. 6).

The Proximal and Distal Rotational Joints 5218, 5219 are identical (FIG. 6). Each includes an Inside Bore Diameter 5204, 5213 that provides concentric alignment with the Main Grip Mating Surface 5105 and the Broach Connect Body Mating Outside Diameter 5605, respectively. Center Body Mating Thrust Surface 5205/Mating Planer Surface 5211 provides a planer surface for coincident joining of Main Grip Thrust Face 5109 and Broach Connect Body Outside Diameter 5602, respectively. Dowel Bore 5206, 5210, perpendicular to the Inside Bore Diameter 5204, 5213 (2 per Rotational Joint) provides a bore for Dowel 5400 (2 per Rotational Joint) to be pressed fit into the respective bores allowing linear connectivity of the Main Grip 5100 and Broach Connect Body 5600, respectively. The dowels may be removed to disassemble the Broach Handle for cleaning. Push Button Bore 5207, 5209 is the mating bore for Push Button 5500 and provides 360-degree rotational capability of the Main Grip and Broach Connect Body 5600, respectively. At the bottom of the Push Button Bore 5207, 5209 is a Conical Spring Bore 5208 which are mating bore pockets for Conical Springs 5300. Base Surface 5212 provides a mating bottom/support surface for Conical Springs 5300.

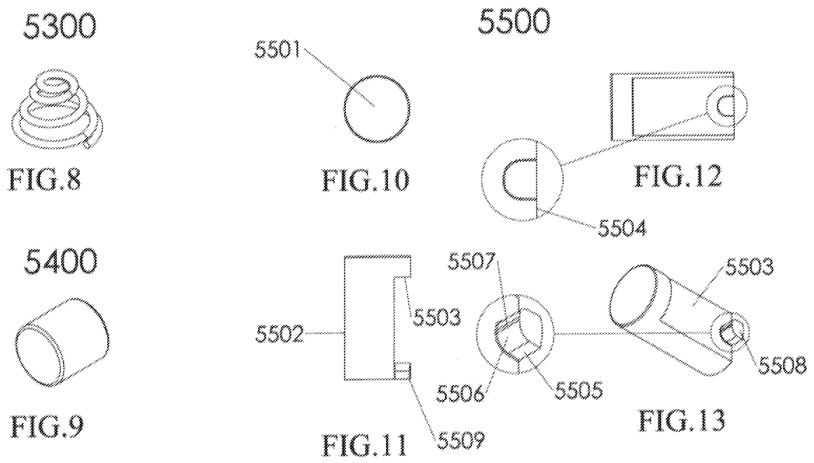

FIG. 8 depicts the Conical Spring that rests in the Conical Spring Bores 5208. The Conical Spring the force to engage and disengage Push Button 5500.

FIG. 9 depicts the 316 Stainless Steel Dowel 5400. Each Rotational Joint employ 2 Dowels. The dowels provide device assembly of the 360-degree rotational connectivity of the Main Grip Connecting Groove 5106 with the Dowel Bore 5206 of the Proximal 360-degree Rotational Joint and 360-degree rotational connectivity of the Dowel Bore 5210 of the Distal 360-degree Rotational Joint with the Broach Connect Body Connecting Groove 5606.

FIGS. 10 through 13 depict the Push Button 5500 that provides manual deployment of the 360-degree Rotational Joints 5218, 5219. The Push Button Top Surface 5501 provides a manual deployment surface for disengagement of the rotational joints. The Push Button Outer Diameter 5502 provides the desired clearance for a slip-fit with Push Button Bores 5207, 5209. Push Button First Planer Surface 5503 provides the desired clearance with Main Grip Outside Diameter 5112 and Broach Connect Body Outer Diameter 5602. The Bottom or Base Planer Surface 5504 provides a mating surface with the Conical Spring 5300. At the bottom of the Push Button is an Engaging Member 5509 for connectivity with the Main Grip Outside Diameter 5112 and Broach Connect Body Outer Diameter 5602. The Engaging Member includes mirrored Angled Locking Surfaces 5505 providing locking geometry mating with Positioning Slots 5108, 5601. Push Button Radial Geometry 5506 provides radial clearance with the radial notch surface of Positioning Slots 5108, 5601. Further, Push Button Second Planer Surface 5507 provides rotational clearance with Positioning Slots 5108 and 5601. Push Button Cylindrical Outer Surface 5508 provides coincident outer diameter with Push Button Outer Diameter 5502.

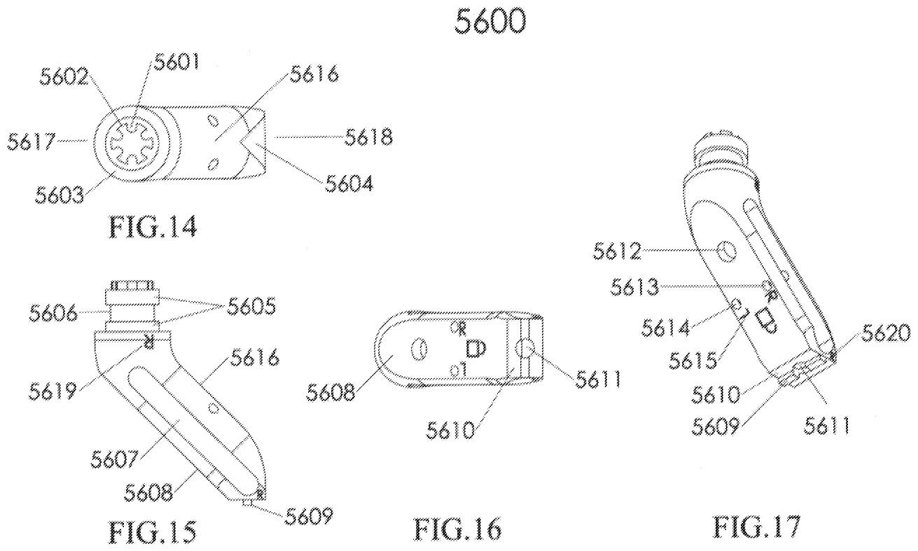

FIGS. 14 through 17 depict the Broach Connect Body 5600. The proximal end 5617 is substantially cylindrical and the distal end 5618 is substantially a rectangular prism. The junction between the proximal end and the distal end creates an angle 5619. In the Neutral and/or Locked position for either offset, Left or Right, at 0° rotation (Neutral), the Main Grip mechanical component being parallel to the patient's torso creates an angle of 54.74° respectively, regarding right and left offset.

The proximal end 5617 is similar in construct as the distal end of the Main Grip. The proximal end includes two Broach Connect Body Mating Outside Diameters 5605 separated by a Broach Connect Body Connecting Groove 5606. Broach Connect Body First Planer Surface 5603 provides coincident planer surface for Center Body Mating Planer Surface 5211. Broach Connect Body Outside Diameter 5602 provides clearance for the Push Button Second Planer Surface 5507 of the Engaging Member 5509 and has six positional slots 5601. The positional slots 5601 engages with Push Button Engaging Member 5505 allowing the Center Body 5200 to rotate with respect to the Broach Connect Body 5600 in 60-degree increments for 360-degree rotation. The positional slots engage with the Push Button Angled Locking Surfaces 5505 of the Push Button Engaging Member 5509. The Broach Connect Body Outer Body Radius 5604 provides the necessary anatomical clearance during use of the Broach Handle. Further, the Broach Connect Body Outer Body 5616 provides the necessary anatomical clearance while maintaining functional strength for unlimited procedures of the Broach Handle.

The distal end 5618 of the Broach Connect Body 5600 includes a Broach Connect Body Planer Surface 5608 which provides the anatomical clearance, weight reduction and rotational thrust support of the Lock Lever Planer Face/Surface 5904. Also, at the distal end 5618 are Left and Right Indicators 5619

(FIG. 15 depicts the Right Indicator). The Broach Connect Body Planer Surface 5608 also helps eliminate bacteria growth between the components during sterilization. Broach Connect Body Slot 5607 provides clearance for Pinchers 6200 and 6300. Broach Connect Body Rotational Stop 5609 prevents rotation of the Broach 6100 via Broach Channel 6008 of Adapter 6000. Broach Connect Body Thrust Face 5610 at the proximal end of the Broach Connect Body 5600 provides a coincident mating surface with Broach Surface 6007 of Adapter 6000. Broach Connect Body Broach Bore 5611 is a connective alignment bore for Broach Pin 6101 on the Broach. Lock Pin Bore 5612 is a connective alignment bore for the Lock Pin 5800. Right Pincher Bore 5613 is a connective alignment bore for the Right Pincher Dowel 5700. Left Pincher Bore 5614 is a connective alignment bore for the Left Pincher Dowel when the Broach is in the Left. The Left and Right Pincher Dowels 5700 are identical and given one reference number. Engraving 5615 provides clarification for left and right Broach Handle positions.

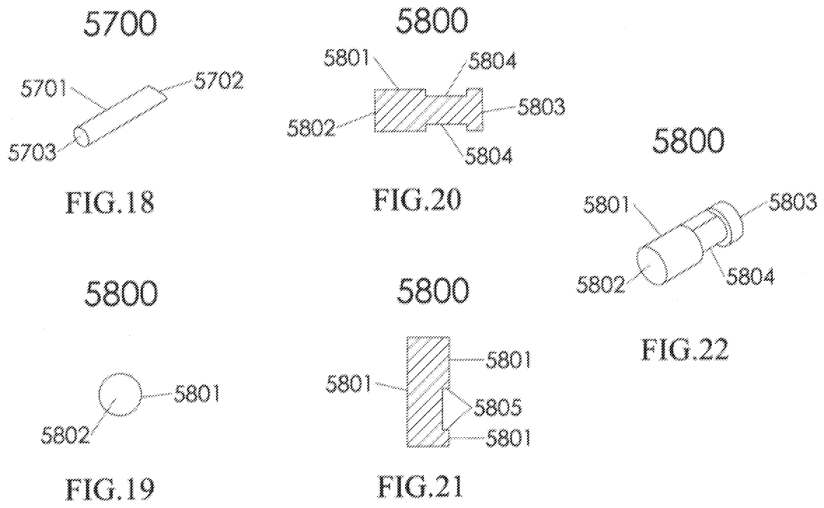

FIG. 18 depicts the Pincher Dowel 5700. The Pincher Dowel provides positional support of the Left and Right Pinchers 6200, 6300. Pincher Dowel Outer Diameter 5701 provides pivotal location of Left Pincher Bore 6206 and Right Pincher Bore 6306. First End Surface 5703 provides a flush or coincident surface with Broach Connect Body Outer Body 5616, eliminating bacterial growth during sterilization. Blend Surface 5702 provides a blended surface with Broach Connect Body Outer Body 5616, eliminating bacterial growth during sterilization.

FIGS. 20 through 22 depict the Lock Pin. The Lock Pin provides rotational engagement and disengagement of Broach 6100 via a mechanical functionality with Left and Right Pinchers 6200 and 6300. Lock Pin Outer Diameter 5801 provides the mating geometry with Broach Connect Body Bore 5612. Lock Pin First End Surface 5802 provides a colinear planer surface with Lock Lever Planer Face/Surface 5904, eliminating bacterial growth during sterilization. Lock Pin Second End Surface 5803 provides a colinear surface with Broach Connect Body via Lock Pin Bore 5612, eliminating bacterial growth during sterilization. Lock Pin Radial Undercut Groove 5804 provides rotational engagement and disengagement of Broach 6100 via mechanical functionality with Pincher Blind Bores 6203 and 6303 depending on if the Broach Handle is in a left or right position. Sides of Radial Undercut Groove 5805 provide the necessary groove width for maintaining a centralized position of either the Left Pincher Outer Body Surface 6201 or the Right Outer Body Surface 6301 within Slot 5607.

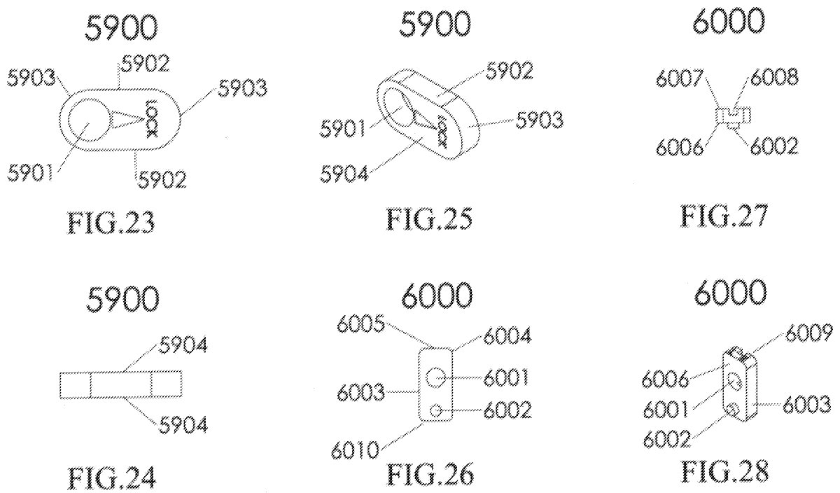

FIGS. 23 through 25 depict the Lock Lever 5900 which provides manual rotational lever for engaging and disengaging Broach 6100. The Lock Pin 5800 is press fit into Lock Pin Bore 5901. The Outer Side Boundary 5902 of the Lock Lever provides for easy manual rotational manipulation. The Top and Bottom Boundary Geometry 5903 (semi-circular) provides safe radial geometry for easy manual gripping. The Planer Face/Surface 5904 of the Lock Lever provides planer surfaces for mating with the Broach Connect Body Planer Surface and the anatomy of the patient.

FIGS. 26 through 29 depict the Adapter 6000 which provides a rotational stop between the Broach Connect Body 5600 and the Broach 6100. The Adapter may have different geometries depending on the manufacturer of the Broach 6100. To compensate for the selection of the Adapter depends on the manufacturer of the Broach, allowing the connectivity of the Broach to the Broach Handle. The Adapter comprises an Adapter Bore 6001 that locates the Broach Forward/Back, Up/Down, Left/Right, Yaw, and Pitch (5 degrees of freedom). The Broach Pin 6101 passes through the Adaptor Bore 6001. The Adapter Bore 6001, the Adapter Boss 6002 and the thickness of the Adapter is specific depending on the manufacturer of the Broach 6100. The Adapter thickness determines the linear position of the Broach Pin 6101 undercut 6102 for the Pincher to lock the Broach into position. Adapter Boss 6002 provides restricted polar alignment or roll of the Broach. The Adapter Outer Side Boundary 6003 provides optimum anatomical clearance. The Outer corner Boundary 6004 provides radial geometry promoting clearance and safety. The Adapter Top and Bottom Geometry 6005 and 6010 provides planer surfaces for desired anatomical clearance and Broach 6100 insertion clearance bonus depth. The Adapter Top 6005 also includes Left and Right Indicators 6009. Adapter First Planer Thrust Face 6006 provides coincident planer thrust or impact surface for the Broach. Adapter Second Planer Thrust Face 6007 provides coincident planer thrust or impact surface for the Broach Connect Body Thrust Face 5610. Adapter Notch or Groove 6008 provides a polar positional stop with Broach Connect Body Rotational Stop 5609.

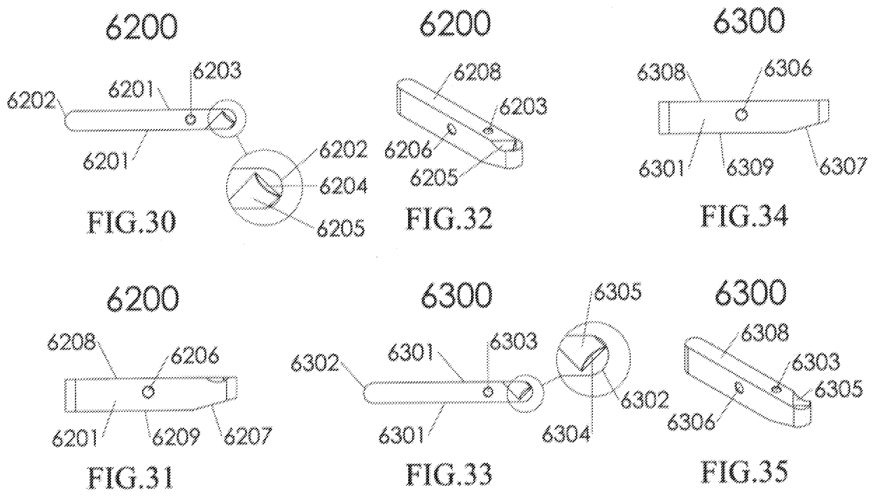

FIGS. 30 through 32 depict the Left Pincher 6200 and FIGS. 33 through 35 depict the Right Pincher 6300. The Left and Right Pinchers manually engage and disengage with Broach 6100. The construction of the Left and Right Pinchers are essentially identical with the exception that they are mirrored opposites of each other.

Depicted in FIGS. 30 through 32 is the Left Pincher 6200 having a Top Planer Surface 6208 and a Bottom Planar Surface 6209. Left Pincher Outer Body Surface 6201 provides the desired parallel boundaries for mating with Broach Connect Body Slot 5607. Left Pincher End Surfaces 6202 provides the desired radial boundaries for mating with Broach Connect Body Slot 5607. Left Pincher Blind Bore 6203 provides a cylindrical pocket for nesting Compression Spring 6400. FIG. 37 Left Pincher Mating Surface Geometry 6204 provides the desired mating surface to connect with Broach 6100, in particular the Broach Pin undercut 6102, thereby providing a mechanical linear motion stop/lock that prevents the Broach from disengaging from the Broach Handle when the Lock Lever 5900 and Lock Pin 5800 are engaged. Left Pincher Bore 6206 provides positioning and rotational functionality via Pincher Dowel

Outer Diameter 5701. Left Pincher Angled Planer Surface 6207 provides the necessary anatomical clearance to the anatomy wound in case of over insertion.

The Left Pincher Bottom Planer Surface 6208 provides the necessary planer surface for the engagement and disengagement of the Broach 6100 via the Lock Pin Outer Diameter Surface 5801 and the Lock Pin Radial Undercut Groove 5804.

Depicted in FIGS. 33 through 35 is the Right Pincher 6300 having a Top Planer Surface 6308 and a Bottom Planar Surface 6309. Right Pincher Outer Body Surface 6301 provides the desired parallel boundaries for mating with Broach Connect Body Slot 5607. Right Pincher End Surfaces 6302 provides the desired radial boundaries for mating with Broach Connect Body Slot 5607. Right Pincher Blind Bore 6303 provides a cylindrical pocket for nesting Compression Spring 6400. Right Pincher Mating Surface Geometry 6304 provides the desired mating surface to connect with Broach 6100, in particular the Broach Pin undercut 6102, thereby providing a mechanical linear motion stop/lock that prevents the Broach from disengaging from the Broach Handle when the Lock Lever 5900 and Lock Pin 5800 are engaged. Right Pincher Bore 6306 provides positioning and rotational functionality via Pincher Dowel Outer Diameter 5701. Right Pincher Angled Planer Surface 6307 provides the necessary anatomical clearance to the anatomy wound in case of over insertion. The Right Pincher Bottom Planer Surface 6308 provides the necessary planer surface for the engagement and disengagement of the Broach 6100 via the Lock Pin Outer Diameter Surface 5801 and the Lock Pin Radial Undercut Groove 5804.

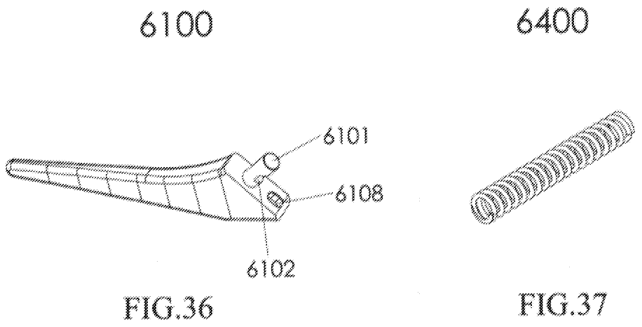

FIG. 36 depicts Broach 6100. Various commercially available broaches may be attached to Broach Handle 5000 to be employed in various hip arthroplasty procedures. The Broach includes a Broach Pin 6101 with a Broach Pin Undercut 6102.

FIG. 37 depict Compression Spring 6400. Compression Spring provides bi-linear expanding forces perpendicular to its axis. That is, there are opposite coaxial extending forces exerted bi-directionally along the Spring's axis and starting at the axis centroid. These expanding forces provide a locking engagement of the Broach via the Pinchers and requires depression of the Pinchers for disengagement of the Broach via the lock lever by rotation. These expanding forces provide a locking engagement with both the Left and Right Pinchers, feature 6204 and 6304 of FIGS. 30 and 33, respectively.

A unique feature of the present invention is that the Broach Handle can articulate with respect to the various sections comprising the Broach Handle. An advantageous feature of this articulation in combination with the bends of the various sections allows for the Main Grip to be offset spatially from the Broach Connect Body yet maintain parallel longitudinal axes. The amount of offset or spatial separation between the Main Grip and the Broach Connect Body section may be adjusted by the amount of articulation (rotation) between the Main Grip and the Center Body, and the Center Body with the Broach Connect Body. This offset is desirable depending on the physical features of the patient. That is, for a slender patient undergoing hip replacement surgery the surgeon does not require as much offset as for a patient who is more rotund about the midsection and hip area. By maintaining parallel but spatially offset longitudinal axes between the Main Grip and the Broach Connect Body, the force delivered by the surgeon onto the Force Disc is transmitted in the same linear direction onto the Broach Connect Body and ultimately the Broach.

Another beneficial feature is that it allows the surgeon to configure the instrument as either a Right or Left Instrument, depending on which hip is being replaced. The Broach Connect Body Engravings 5615, 5619 and 5620 together with Adapter Indicator 6009 and the Center Body Engravings 5203 aid the surgeon in identifying the particular configuration of the Broach Handle. As depicted in FIG. 41, the Broach Handle is in a Left Instrument configuration with the Main Grip in a neutral position. As shown in FIG. 41, Center Body Engraving 5203 and identify a Left configuration. This is also shown by Left and Right Indicators 6009 of the Adapter 6000 with Broach Connect Body Engraving 5620. Similarly, FIG. 42 depicts the Broach Handle in a Right Instrument configuration with the Main Grip in a neutral position. By depressing the Push Button 5500 associated with the Distal Rotational Joint 5219 of the Center Body, the surgeon can align the left and right offset positions by aligning the Center Body Engraving 5203 with the Broach Connect Body Engraving 5619 so that the same letterings align. This is also shown by Left and Right Indicators 6009 of the Adapter 6000 with Broach Connect Body Engraving 5620. The Adapter Indicator 6009 also provides indication for the left and rightBroach 6100 attachment.

Depending on the physical characteristics of the patient, a surgeon may need to offset the Force Disc of the Broach Handle from the linear axis of the Broach. As can be seen in FIGS. 41 and 42, the longitudinal axis of the Broach 6100 is parallel to the longitudinal axis of the Main Grip, allowing a surgeon to apply force to the Main Grip and have this force transmitted in the same direction onto the Broach 6100. This configuration is a double offset configuration. Various amount of offset is possible. That is, for a slender patient undergoing hip replacement surgery the surgeon does not require as much offset as for a patient who is more rotund about the midsection and hip area. The offset is accomplished by articulating/rotating both the Main Grip and the Broach Connect Body via the Push Button mechanisms affording a surgeon to configure the Broach Handle that will allow the surgeon to operate, for example, on the medullary canal by exerting a force onto the Broach Handle by striking the Force Disc of the Main Grip. By maintaining parallel but spatially offset longitudinal axes between the Main Grip and the Broach, the force delivered by the surgeon onto the Force Disc is transmitted in the same linear direction onto the Broach.

FIG. 44 depicts the Adapter Function Chart and the six degrees of freedom (forward/back, up/down/left/right, yaw, pitch and roll). Six degrees of freedom (6DOF) refers to the freedom movement of a rigid body (e.g., Adapter) in three-dimensional space. Specifically, the body is free to change position as forward/backward (surge) up/down (heave), left/right (sway) translation in three perpendicular axes, combined with changes in orientation through rotation about three perpendicular axes, often termed yaw (normal axis), pitch (transverse axis), and roll (longitudinal axis).

The aspects and embodiments of the present invention provide a method for improving the preparation of the bone for example the femur in a hip replacement surgery. As discussed, the articulating broach handle allows for quick and simple adjustments to the broach handle to accommodate for whether the surgeon is performing a right hip replacement or a left hip replacement, and the physical stature of the patient (protruding midsection or not).

Depending on if the surgeon will be performing a left hip or right hip replacement, the surgeon may need to articulate and configure the Broach Handle as either a left or right handle. Depending on the physical stature of the patient, the surgeon can increase or decrease the offset by rotating the three main sections of the Broach Handle 5000, Main Grip 5100, Center Body 5200 and Broach Connect Body 5600 with respect to each other using the Push Button mechanisms (Push Button 5500, Conical Spring 5300, Stainless Steel Dowel 5400).

Method of Use

The Broach Handle 5000 of the Fifth Embodiment allows a surgeon to configure the Broach Handle for a right lateral offset broach or left lateral offset broach. Using the Broach Handle of the Fifth Embodiment, a surgeon will select a Broach 6000 from one of the available manufacturers. Depending on the manufacturer of the Broach, the surgeon will select the appropriate Adapter 6100. The Broach is connected to the Broach Connect Body 5600 via the Adapter 6100 and is secured in place as discussed above.

Depending on the physical characteristics of the patient, the surgeon may need to offset the Force Disc of the Broach Handle from the linear axis of the Broach. The surgeon may adjust/swing/articulate the Main Grip and the Broach Connect Body with respect to each other and to the Center Body, offsetting the Force Disc for either a right lateral offset or a left lateral offset. This is accomplished by adjusting the various sections with respect to one another such that the angled surfaces are in contact witheach other. The relative positions of the sections are maintained until the surgeon engages the articulating mechanisms (Push Buttons) to adjust one section (Main Grip, Center Body, Broach Connect Body) with respect to the other section. The ability to articulate the various sections of the Broach Handle with respect to one another allows the Broach Handle to be used on either the left or right side.

Through the use of the Adapter 6100, broaches from different manufacturers may be used by the present invention.

Although the present invention has been described in considerable detail with reference to certain embodiments thereof, other embodiments are possible. Therefore, the spirit and scope of the appended claims should not be limited to the description of the embodiments contained therein. It should be understood that various changes, substitutions, additions and alterations can be made by one skilled in the art without departing from the spirit and scope of the invention as defined by the appended claims. Moreover, the scope of the present application is not intended to be limited to the particular embodiment(s) of the device, process, machine, manufacture and composition of matter, means, methods and or steps described in the specification. As one of ordinary skill in the art will readily appreciate from the disclosure herein, processes, machines, manufacture, composition of matter, means, methods or steps, presently existing or later to be developed that perform substantially the same function or achieve substantially the same result as the corresponding embodiments described herein may be utilized according to the present invention.

1. An articulating surgical tool handle, comprising:
a force disc;
a main grip;
a center body;
a broach connect body;
an adapter;
first and second articulating means;
means for releasably attaching a tool,
wherein the adapter allows a tool to attach to the articulating surgical handle,
wherein the first and second articulating means enable the center body to rotate in discrete increments, within a range of rotation between 0 degrees and 360 degrees, about a longitudinal axis of the main grip and the broach connect body, respectively.

2. The articulating surgical tool handle as claimed in claim 1, wherein the first articulating means connects the main grip and the center body and the second articulating means connects the center body and the broach connecting body.

3. The articulating surgical tool handle as claimed in claim 1, wherein the main grip, center body, broach connect body connected to the tool and the first and second articulating means allow for a double offset configuration.

4. The articulating surgical tool handle as claimed in claim 3, wherein in the double offset configuration, the main grip is offset spatially from the tool and maintaining parallel axes of the longitudinal main grip and broach connect body.

5. The articulating surgical tool handle as claimed in claim 4, wherein the spatial offset of the main and broach connect body is adjustable by the first and second articulating means.

6. The articulating surgical tool handle as claimed in claim 1, wherein the discrete increments are 60 degrees of rotation.

7. The articulating surgical tool handle as claimed in claim 1, wherein the tool is a broach.

8. The articulating surgical tool handle as claimed in claim 1, wherein each of the first and second articulating means comprises a push button and spring,
wherein the push button of the first articulating means allows for the adjustment of the position of the main grip and center body relative to each other, wherein the push button of the second articulating means allows for the adjustment of the position of the center body and the broach connect body relative to each other.

9. The articulating surgical tool handle as claimed in claim 1, wherein the Adaptor has a thickness, a bore and a boss.

10. The articulating surgical tool handle as claimed in claim 9, wherein the Adaptor is selected from a multitude of adapters having different geometries regarding its thickness the location of the bore and boss.

11. An articulating surgical tool handle, comprising:
a force disc;
a main grip;
a center body;
a broach connect body;
an adapter having a thickness, bore and boss;
first and second articulating means;
means for releasably attaching a tool,
wherein the adapter allows different tools to attach to the articulating surgical handle.

12. The articulating surgical tool handle as claimed in claim 11, wherein the Adaptor is selected from a multitude of adapters having different geometries regarding its thickness the location of the bore and boss and where selection is based on the tool to be used.

13. The articulating surgical tool handle as claimed in claim 12, wherein the tool is a broach.
